(19) United States
(12) Patent Application Publication
(10) Pub. No.: US 2009/0087925 A1
(43) Pub. Date: Apr. 2, 2009

(54) DEVICES AND METHODS FOR ANALYSIS OF SAMPLES WITH DEPLETION OF ANALYTE CONTENT

(75) Inventors: Peter Wagner, Belmont, CA (US); Frank Zaugg, Belmont, CA (US); Mark Mitchnick, East Hampton, NY (US)

Correspondence Address: King & Spalding LLP, P.O. Box 889, Belmont, CA, 94002-0889, US

(73) Assignee: Zyomyx, Inc., Hayward, CA (US)

(21) Appl. No.: 11/865,555

(22) Filed: Oct. 1, 2007

(51) Int. Cl. G01N 33/543; B01J 19/00; C12M 1/00; G01N 21/00; G01N 33/00

(52) U.S. Cl. 436/518; 422/68.1; 435/287.1; 422/82.05; 436/63

(57) ABSTRACT

A system and method for determining the presence and/or concentration of one or more analytes in a sample that comprises a fluid, the system comprising a solid substrate comprising a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path or paths, the flow path or paths being designed so that the one or more analytes are depleted from the sample and bound in a non-linear manner to the portion of the flow path or paths containing immobilized capture agent or agents, producing an analyte depletion end region for each analyte between the beginning and the terminus of the analyte determination flow path.

### CROSS-REFERENCES TO RELATED APPLICATIONS

This application claims the priority of U.S. Provisional patent application 60/848087 filed Sep. 29, 2006 entitled “Novel Cell Count Microdevice for HIV Monitoring”.

### BACKGROUND OF THE INVENTION

This invention relates to systems and methods for determining whether one or more analytes are present in a sample that comprises a fluid and/or quantifying such analyte(s) and/or determining the concentration of such analyte(s) in the sample. More particularly, as compared to conventional devices for this purpose, which depend on relatively complex multi-step protocols such as sandwich immunoassays, special reagents, special materials of construction and the like, the systems and methods according to this invention can detect and/or quantify analytes in a manner that allows relatively simple and rapid assessment of analyte concentration, employing the principle of depletion and non-linear capture of analyte content in the sample.

In some embodiments the devices and methods of this invention are useful for identifying HIV-positive patients and monitoring their immune status through determining the level of CD4⁺ lymphocytes (“CD4 count”) in blood samples below and above the clinically relevant threshold.

To date, counting the number of CD4 lymphocytes in blood is considered the most accurate method for evaluating and monitoring the clinical stage of HIV infection. The United States Public Health Service has recommended that CD4 cell levels in people with HIV should be tested on a regular basis in order to make decisions about clinical needs such as antiretroviral therapy and for evaluation of treatment efficacy. Although several CD4 count techniques are currently available (such as two-color flow cytometry and microcytometry), there is a great need for simpler and cheaper CD4 counting assays.

In developed countries, CD4 count assays are carried out routinely in people known to be infected with HIV. However, for resource-poor countries, current CD4 count technologies are too expensive and too complex in operation and thus not suitable in most settings. There is a lack of access to such routine laboratory tests, which could be used for millions of people infected with HIV who would benefit from CD4 count monitoring. At a minimum, especially for use in such locations, CD4 tests need to be inexpensive, easy to operate and read by minimally educated personnel and without the use of expensive instruments, suitable for storage at room temperature with a long shelf life and ideally free of the need for external reagents and associated handling operations. Unfortunately, no existing technology meets all these requirements.

The need for a fully integrated test free of external reagents, similar to a pregnancy test strip or the like, excludes complex assay formats such as sandwich immunoassays (which have too many solutions operating in sequence), or bead-based assays (which tend to have a short shelf life at elevated temperatures, as well as handling issues). In addition, the preference for no or minimal instrumentation rules out complex optical detection schemes such as fluorescence, surface plasmon resonance and diffraction-grating measurements (instrumentation for these is either too expensive, bulky or often subject to false-positive results).

### BRIEF SUMMARY OF THE INVENTION

In one aspect the invention comprises a system for determining one or more analytes in a sample that comprises a fluid, the system comprising a solid substrate having a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path or paths, the flow path or paths being designed so that the one or more analytes are depleted from the sample and bound in a non-linear manner to the portion of the flow path or paths containing immobilized capture agent or agents, producing an analyte depletion end region for each analyte between the beginning and the terminus of the analyte determination flow path.

In another aspect the invention comprises a method for determining the presence and/or concentration one or more analytes in a sample that comprises a fluid, the method comprising introducing the sample into a system or device comprising a solid substrate having a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path, the flow path or paths being designed so that the one or more analytes are depleted from the sample and bound in a non-linear manner to the portion of the flow path or paths containing immobilized capture agent or agents, producing an analyte depletion end region for each analyte between the beginning and the terminus of an analyte determination flow path, causing the sample to flow through the flow path and, based on the analyte depletion end region, determining the presence and/or concentration of said analyte or analytes in the sample.

### BRIEF DESCRIPTION OF THE DRAWINGS

FIG. 1 is a generalized depiction of the systems of this invention.

FIG. 2 depicts various embodiments of the flow paths that may be used in the devices.

FIG. 3 depicts additional embodiments of the flow paths.

FIG. 4 depicts some optional features of devices according to the invention.

FIG. 5 depicts one embodiment of the invention that is particularly suitable for testing for HIV.

FIG. 6 depicts the device of FIG. 5, showing a typical test result.

FIG. 7 depicts a series of test results using portions of a device as in FIG. 5 showing depletion end regions for several different analyte concentrations.

FIG. 8 shows another portion of such a device in which the depletion end region is displayed using a different technique.

### DETAILED DESCRIPTION OF THE INVENTION

In one aspect the invention comprises a system for determining one or more analytes in a sample that comprises a fluid, the system comprising a solid substrate having a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path or paths, the flow path or paths being designed so that the one or more analytes are depleted from the sample and bound in a non-linear manner to the portion of the flow path or paths containing immobilized capture agent or agents, producing a detectable analyte depletion end region for each analyte between the beginning and the terminus of its respective analyte determination flow path (i.e., that flow path or portion of a flow path that contains a capture agent for that analyte).

In another aspect the invention comprises a method for determining the presence and/or concentration of one or more analytes in a sample that comprises a fluid, the method introducing the sample into a system or device comprising a solid substrate having a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path, the flow path or paths being designed so that the one or more analytes are depleted from the sample and bound in a non-linear manner to the portion of the flow path or paths containing immobilized capture agent or agents, producing an analyte depletion end region for each analyte between the beginning and the terminus of an analyte determination flow path, causing the sample to flow through the flow path and, based on the analyte depletion end region, determining the presence and/or concentration of said analyte or analytes in the sample.

Devices and methods using the principles of this invention afford simple, fast and accurate measurements in the absence of external reagents, although the use of external reagents is not outside the bounds of this invention. In some embodiments they may possess a long shelf life even at elevated temperatures, do not require external sample preparation steps, are easy to use without extensive training, and require no or at most minimal instrumentation. For these reasons, they are well suited for use in detecting and monitoring persons having diseases or conditions in resource-poor areas, including areas that experience relatively high ambient temperature, and in which highly trained personnel are scarce. However, while these features are possessed by some embodiments of this invention, the invention is not limited to such devices. For example, devices that rely on more complicated, even automated, instrumentation, are also encompassed within the scope of this invention, so long as they posses the necessary features, for example the use of analyte depletion assay and binding techniques as described herein. Such devices are useful in the determination and monitoring of large populations of subjects.

As a result of designing the devices according to the invention for simplified operator handling and instrumentation, assay complexities are transferred to the “inner parts” of the test device. Some of the most promising embodiments involve a high degree of surface-based phenomena, yet are easy to use.

In its most basic essence, the invention relates to systems that include sample analysis devices comprising a sample inlet connected to a depletion flow path with a defined start and endpoint. The flow path contains one or more analyte capture layers or regions that are designed so as to effectively and progressively deplete one or more analytes in a sample traveling through the flow path, leading to a non-linear capture of analyte (as defined herein) and a depletion end region within the flow path for each analyte. The distance of the depletion end region from the start point is in a specific pre-calibrated relationship to the concentration of the analyte. The dimensional properties of the flow path are varied so as to design a device for a given analyte or analytes that includes a readout location of the analyte depletion region(s) relative to the beginning of the flow path that, in addition to establishing the presence of analyte(s) in the sample, can also provide information on the initial concentration of the analyte(s) in the sample. The general detectability of the analyte(s) is also shown at that readout location. In addition to the devices the system may also include detectors, for example instruments, for detecting the location of the analyte depletion end region or regions.

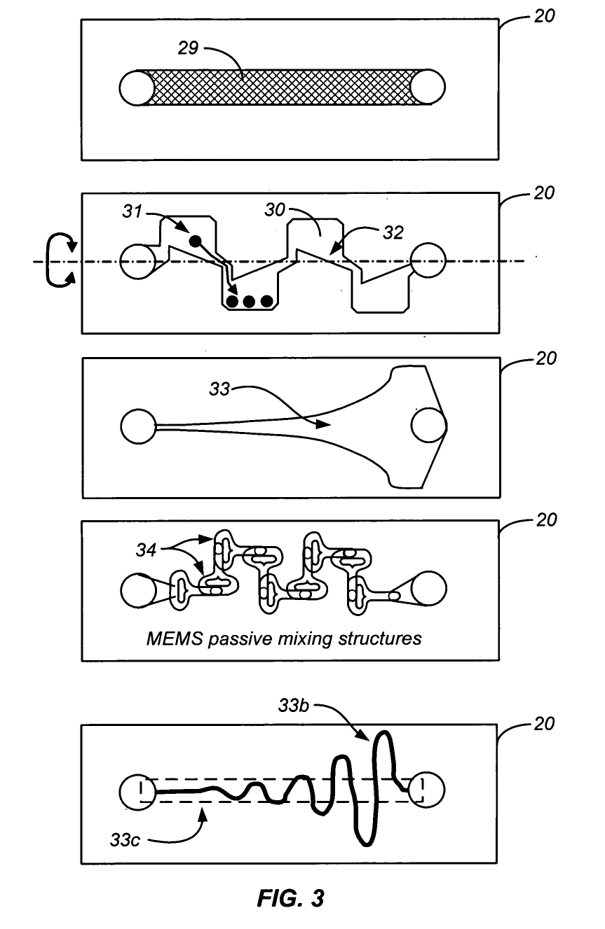

The systems, devices and methods of this invention function through depletion of the analyte from the sample onto the surface of the flow path and binding of it to that surface in a non-linear manner. The term “non-linear analyte depletion” refers to a decrease in the density of captured analyte that becomes non-linear and is achieved within a specified assay time-window, measured over the depletion flow path length from start to end, the flow path length having been designed for and calibrated to be sensitive to a specific concentration range of the analyte when concentration is to be determined. In the systems and method of this invention initially the analyte typically is captured relatively uniformly along the flow path, but at a location in the flow path (hereinafter referred to as the “analyte depletion end region”) the capture becomes non-linear, with a significant drop in the amount of analyte bound to the flow-path surface. The “analyte depletion length”, which may refer either to the length of the flow path that contains captured analyte, or to the overall area of the flow path that contains captured analyte, is either directly or indirectly readable and may then be compared to a calibrated table or ruler indicating depletion length versus estimated concentration of the unknown or known analyte in the sample. The use of area (as opposed to the length) of the flow path that contains captured analyte for this determination may occur, for example, when the system contains a non-linear bending flow path as depicted in FIG. 3, 33b, with a reduced flow path-readout window.

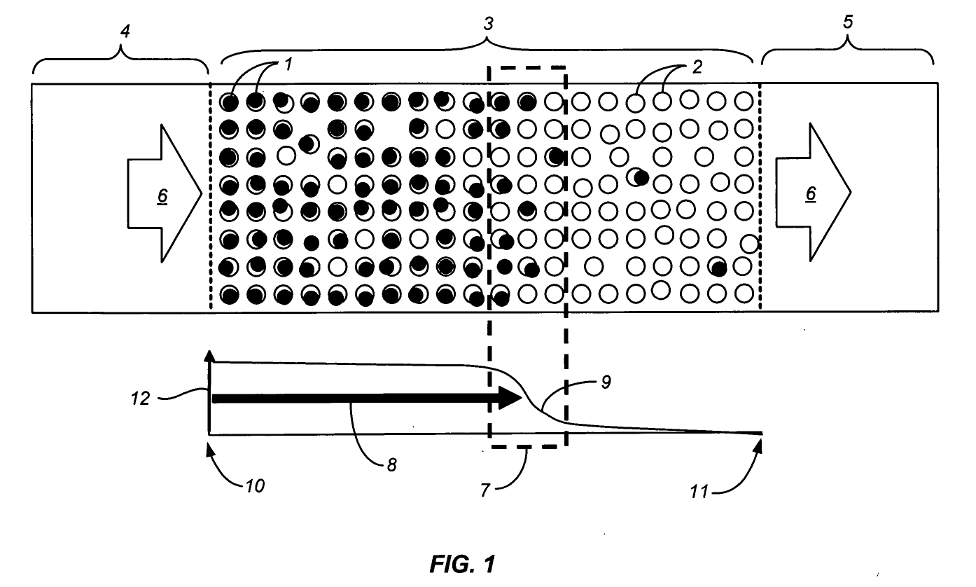

FIG. 1 depicts a generalized system according to this invention. As shown in FIG. 1, a sample is passed along flow path 3 from an upstream processing zone 4 to a downstream processing zone 5. The flow path contains a plurality of analyte capture agents 2 as described hereinafter that capture analyte (indicated as 1) in a non-linear manner. By “capture in a non-linear manner” is meant that initially the analyte typically is captured relatively uniformly along the flow path, but that at a location in the flow path (hereinafter referred to as the “analyte depletion end region”) the capture becomes non-linear, with a significant drop in the amount of analyte bound to the flow-path surface. This is illustrated in FIG. 1, graph 12. The analyte can be labeled or probed with a label, as described below, so that its depletion can be detected along the flow path, and more particularly at the analyte depletion end region 7. The flow path, also referred to as the “analyte determination flow path” has a defined beginning and a defined terminus or end, where the beginning of the flow path is considered to be the location within the system in which the analyte, after any pretreatment steps, enters a portion of the system that contains capture agent, and the terminus or end of that flow path is considered to be the location at which the sample no longer encounters capture agent. The extent of the flow path that contains immobilized capture agent can vary widely, and can constitute less than half of the flow path length or surface, but preferably, at least a substantial portion of the flow path contains a capture agent or agents. Most preferably a major portion of the flow path will contain capture agent and, in some embodiments, the entire flow path will contain capture agent.

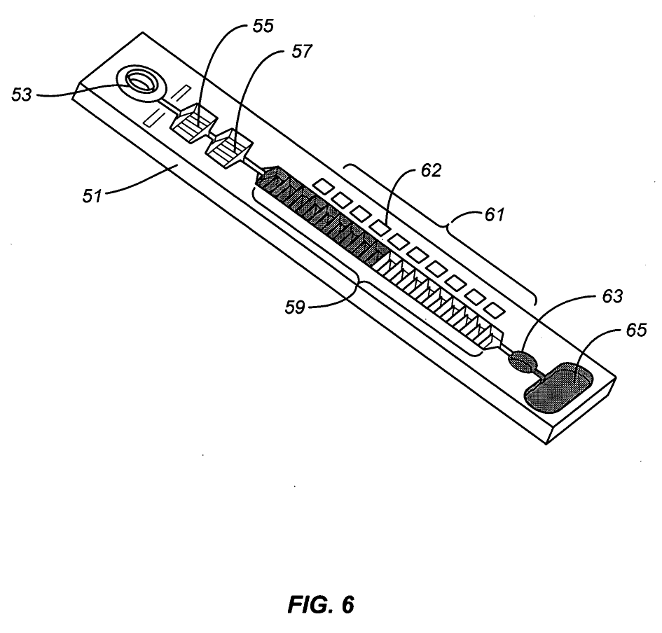

FIG. 1 also contains a graphical depiction 12 showing the amount of bound analyte versus the flow path length. The depletion end region 7 in the graph will exhibit the end of the depleted analyte in a manner that is easily read either by the unaided eye or by an instrument, with or without further staining, depending on the type of analyte tested. Such a result is also depicted in FIG. 6.

The sample can be any liquid, gas or fluid within which one or more specific analytes are to be detected and/or quantified. The analyte may be dissolved or suspended in the fluid, or may be in an emulsion with the fluid. Typical samples include bodily fluids and biopsy or autopsy samples (e.g. blood, blood plasma, blood serum, spinal fluid, joint fluid, eye fluid, feces, urine, saliva, nose-run, tears, sweat, extracted organs, cell slurries or tissue culture supernatants), or fluids extracted or prepared from animals, plants, food, microorganisms or cell cultures. Usable samples also include any liquid, gas or extracted sample obtained in nature (e.g. water samples), or from an industrial or home setting.

The analyte or analytes may be a fluid (liquid or gas), a solid, emulsified, dissolved or suspended material or cellular material. Typical analytes include proteins, antibodies, enzymes, antigens, (poly)peptides, DNA, RNA, lipids, oligonucleotides, cholesterols, sugars, toxins, hormones, messenger molecules, small chemical molecules such as pharmaceuticals and pesticides, as well as macromolecular species such as pollen, whole cells, parts of cells, cell organelles, bacteria, viruses, nanoparticles and pollutants.

The devices of this invention can be built of any suitable material known in the art for making diagnostic or fluidic devices. Preferably, some components of the depletion flow-path are made by injection molding or bonding of polymeric materials (e.g. polystyrene, COC, COP, polycarbonate, or polypropylene). Alternatively, such structures can be created by embossing (polymers) or by various etching/microlithography or micromachining methods (e.g., applied to glass or silicon or other inorganic materials). Suitable structures also can be made by bonding several layers of, e.g., stamped or laser-cut or non-treated thin material foils or by using photopolymer-patterned laminates. Other materials that are known for use in such devices and may be employed in making the devices of this invention are mentioned in, e.g., U.S. Pat. Nos. 6,576,478 and 6,682,942, which are hereby incorporated herein to the extent that their disclosures are not inconsistent with the disclosure herein, and include metals such as gold, platinum, aluminum, copper, titanium and the like, silicon, silica, quartz, glass, and carbon. The devices of this invention can also be composed of other flow path-forming structures, such as tubes and capillaries stretching linearly or bent in a 3-dimensional form, e.g., a capillary tube bent into a spiral.

The flow path for the sample can be a straight path, or it can include curved sections or be composed of curved sections only (e.g., a meandering or serpentine structure). The flow path can also be a vertical channel. The flow path can be a generally open channel or a series of open channels or, alternatively it can be made of a porous material such as nitrocellulose, porous silicon, polymer networks, gel, etc. Again alternatively, the flow path can be composed of a series of chambers that are, or can be placed, in fluid contact with each other. In another embodiment, the flow path can be made of individual flow segments which are separated from each other by structures which can be opened to allow the sample to sequentially move from one segment to the other.

The depletion flow path area can also consist of a plurality of flow path segments arranged in parallel or layered on top of each other, or in another arrangement relative to each other. For determining multiple analytes, the device may contain a plurality of flow paths for the sample. These may be arranged in parallel or in any other convenient manner. For determination of two or more analytes with parallel flow paths, the sample is preferably introduced though a single inlet and removed or collected in a single outlet or downstream chamber, both connected to all of the flow paths. However, a device according to the invention can have multiple injection sites or entry ports, and multiple exit ports or collection chambers, for samples to be analyzed in parallel or for other purposes as described herein. Each of the plurality of flow paths can contain capture agents for different analytes to be determined or the flow paths may serve different purposes. For instance, one flow path may be used to analyze a sample while another may serve for simultaneous calibration. In another embodiment the flow path comprises a series of chambers through which the sample flows, with different chambers containing capture agents for different analytes. For determination of two or more analytes, it is also possible to utilize a single flow path that contains capture agents for different analytes in different portions of the flow path, so that a first analyte can be detected in an upstream segment of the flow path, a second in a middle segment and a third in a downstream segment, for instance. Such an arrangement can be used, though it may require a larger overall device than a device with parallel channels. However, if size of the device is not a significant factor, this embodiment can be quite useful.

The flow path may include structures that improve the mixing of the liquid or enhance or make more frequent the contact of analytes in solution with the capture agent. Embodiments of such structures include passive mixing structures, active mixing elements such as ultrasonic transducers, and MEMS-style mixers.

The flow path can further include structures that increase the surface area containing the capture species. Embodiments of such structures include micro-or mini-pillars, 3-dimensional protruding structures such as macroporous gels, macroporous hard materials such as porous silicon and 3-dimensional nanotube structures composed of various materials, increased surface roughness such as an embossed topography, 3-dimensional polymer networks or structures such as polymer brushes, thin porous layers such as nitrocellulose membranes, sintered spheres of silica or other suitable materials, and bead-loaded flow-path sections.

The flow path preferably is structured such as to maximize the probability that the analyte encounters the capture species in the flow path many times on its travel through the flow path, and also to allow sequential or quasi-sequential depletion of molecular species or other analytes. For example, flow paths in the form of channels are preferably structured such as to provide at least one narrow dimension, and more preferably two (e.g. path width and depth) such that molecules quickly and repeatedly hit the flow-path surface, e.g., by diffusion. Examples of structures having such properties include 3-dimensional open-pore material with pore dimensions in the range of 100 nm to 100 μm, channel-like structures with at least one channel dimension in the range of 500 nm to 500 μm (e.g. channel depth), and multi-pore structures

For example, one embodiment of the invention contains channels which are 150 μm wide and deep and 600 mm long. Another embodiment contains channels 200 μm wide, 25 μm deep and 1000 mm long. Another embodiment uses a 500 μm-thick nitrocellulose membrane as the flow path.

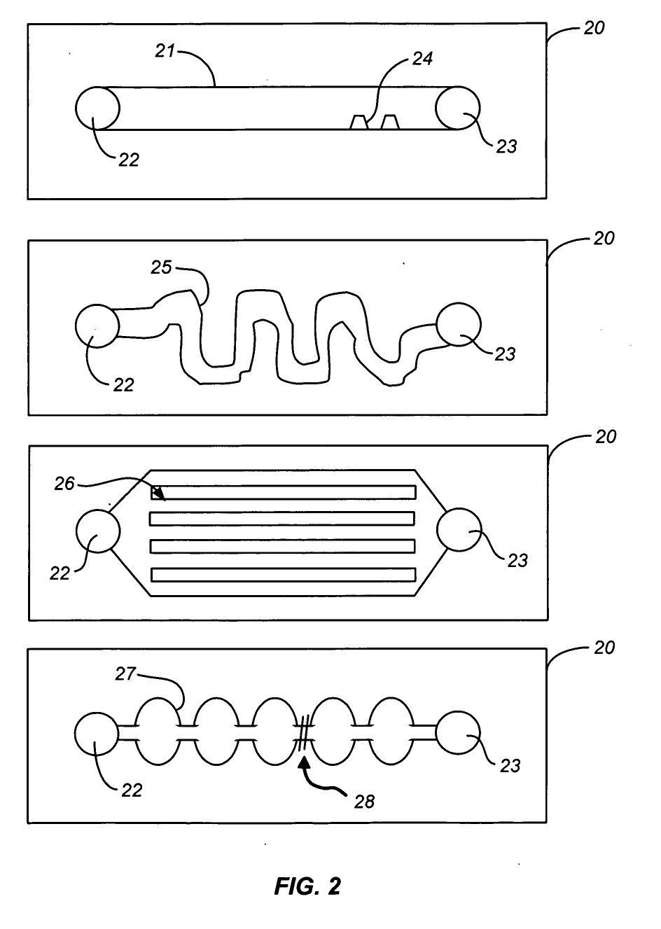

Some examples of flow-path embodiments that may be used in the devices and methods of this invention are seen in FIGS. 2 and 3.

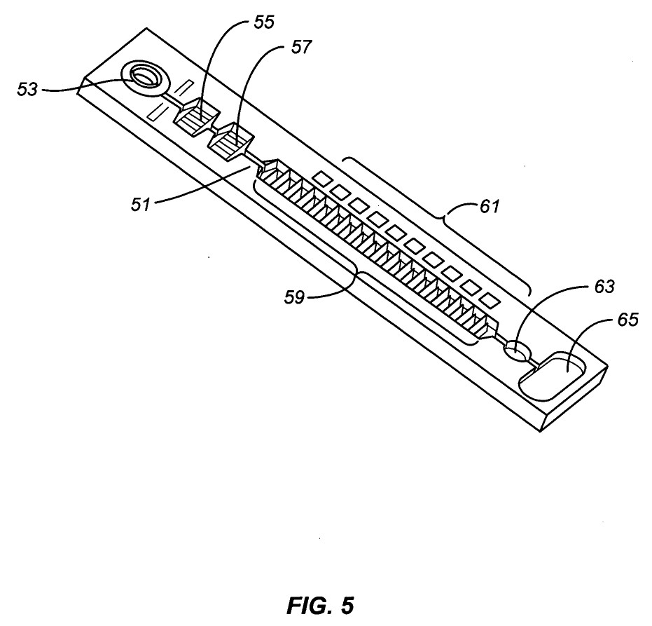

In FIG. 2, 20 indicates the overall general device, 22 indicates a sample inlet, and 23 a sample outlet or means for collecting spent sample. In one embodiment the flow path 21 is a straight channel, designed as described above, and coated with a capture agent for an analyte. Preferably single-channel devices of this type are used to analyze for a single analyte, although, as described above, they may be used to determine two or more analytes. The channel optionally contains three-dimensional structures, represented by 24, to increase the channel surface area, improve fluid mixing, reduce the flow rate or reduce the effective pore size. Another type of flow path, a meandering or serpentine channel, is shown as 25. This type of flow path enables the device to include a relatively long sample flow path in a relatively small device. One example is in the devices shown in FIGS. 5-8.

In FIG. 2, 26 depicts parallel multiple flow paths, which may be open or closed channels or porous material, connected to a common sample inlet and common outlet or collection means. These flow paths optionally contain the types of structures mentioned above to enhance contact, mixing and the like. This embodiment of the invention may be used for analysis of a plurality of analytes, by having each channel contain a capture agent for a different analyte. Alternatively one or more of the parallel channels may be used for calibration and/or for references and/or controls. In the same Figure, 27 depicts a series of interconnected chambers that form the flow path. Optionally the flow path contains one or more active or passive valves 28 between chambers that can be opened at specific moments during the assay, and serve for example, the purpose of preventing backflow of sample or to provide longer residence times leading to improved depletion capture of the analyte to the capture surface. Again, as described above, such an embodiment can be used to determine a single analyte or a plurality of analytes.

In FIG. 3, flow path 29 is defined or filled with a porous material, as described above. Flow path 30 comprises a series of chambers that are not in a straight trajectory, and is particularly useful for analyzing a sample that contains an analyte (indicated as 31) that tends to sediment under gravity. Here the chambers are connected by inclined passageways so that the device can be rotated or turned over to propel the analyte from chamber to chamber with minimal blockage or sample backflow. The passages connecting the chambers may contain capture agents.

Flow path 33a has a non-constant channel cross-section, for instance to increase the dynamic range of the device. The same can be achieved e.g. by a non-linear bending flow path as depicted in 33b, and providing a reduced flow path-readout window as schematically shown in 33c. Flow path 34 depicts MEMS mixing structures as known in the microfluidics art that may be integrated in series, in parallel or in an array, or randomly placed in a flow path, or may they even constitute the flow path.

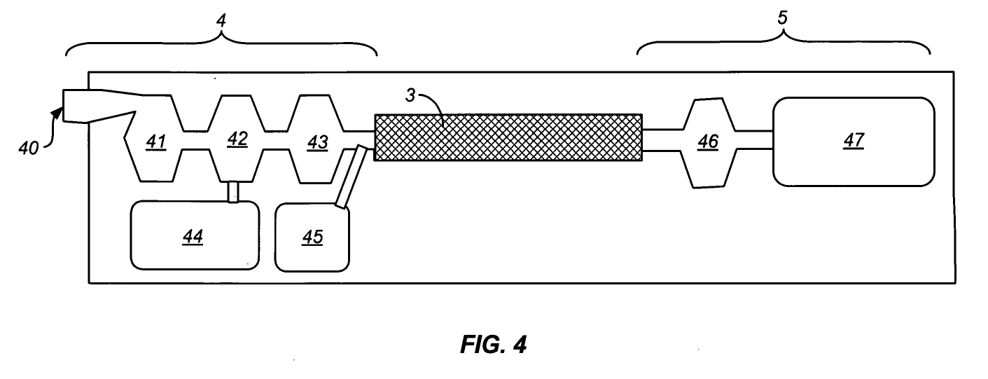

FIG. 4 depicts some optional features that may be present in the zones upstream and downstream of the flow path.

The upstream sample processing zone will include some means for introducing the sample into the device. This can include a sampling device, e.g., a finger prick needle to sample blood, a sample injection septum port, or a sample injection cavity. Another optional upstream feature is a structure used to meter or dose a specific sample volume to be passed through the depletion flow path (FIG. 4, 41). Such a feature could include a defined volume injection structure similar to a syringe or pipette or a microfluidic overflow sampling compartment allowing excess liquid to go into, e.g., an overflow compartment. Other possible upstream sample processing structures may include areas designed for sample pre-treatment, diluting, concentrating, pre-fractioning or filtration (41), areas designed to remove undesired molecular or cellular species in the sample that could interfere with the device principle (43) (e.g., a pre-chamber with immobilized capture agents to specifically capture interfering substance(s)), or a capture layer located behind a dialysis membrane to selectively only capture or remove molecular species of a defined size.

The devices according to this invention can further comprise other reagent or fluid compartments that contain reagents or solvents required for carrying out the assay. These compartments may be in liquid contact with the depletion flow path or may be controlled by passive or active valves. Such optional upstream features shown in FIG. 4 include a sample labeling zone (42), a reagent reservoir (44), and a secondary reagent or pre-wetting fluid reservoir (45). Devices according to the invention can include any or all of the optional features shown in FIG. 4, or may include none of them. Other optional items that may be included in the devices of the invention include barcodes or other identifying labels, company logo, expiration date, a shelf life/storage conditions label, and sensors that indicate whether devices have been exposed to certain environmental conditions (e.g. elevated temperature or humidity conditions, etc).

Preferably, the quantity of the sample introduced into the device is kept to a certain volume for best results. This can be achieved by means such as streaming the sample through the device for a specific time at a specific flow rate, designing a limited and reproducible suction capacity into the device (using e.g. a defined size of a capillary-action suction pad), initially injecting a defined sample volume into the test strip, or active metering of a defined liquid volume via valves, pumps, or flow regulators, and associated electronics.

FIG. 4 also shows features that typically will be contained in the device downstream of the flow path. Devices will typically contain one or both of a positive or negative control area (46) that indicates that the device is working satisfactorily. Typically a positive control area will contain an indicator that the sample has flowed through the device, for instance a substance that changes color or becomes colored when contacted with the analyte carrier fluid. A negative control area will indicate that the sample has not flowed properly through the device. The device may also contain a waste reservoir (47) to prevent physical contact of the user with the sample and allow safe disposal. Alternatively, instead of the reservoir the device may contain an exit port through which depleted sample can be removed from the device. The device may also contain a sucking pad to propel the sample through the flow path or paths.

The propulsion of the sample through the flow path or paths can be achieved via various methods. These include passive propulsion, gravity-based movement of the fluid in the desired direction, capillary action provided by appropriate flow-path dimensions with appropriate wetting properties, or by having the sample flow driven by a capillary action material such as an absorptive wick (e.g. filter paper or advanced suction materials or coatings). The wick can either be positioned at the end of the flow-path or the flow path can itself be constituted of a wicking material or other structures that create a capillary action within the flow-path. For depletion of macromolecular or particulate species, the flow path(s) can also be structured such as to, e.g., use gravity to propel the sample. Flow paths of this type can be constituted of several chamber-like structures which are contacted with each other by liquid bridges. Gravity is used to move the particles from one compartment to the other, sequentially. See, e.g. FIG. 3, 30-32. Alternatively, an evaporative pad can be used to pull liquid through the device by the controlled evaporation of liquid in a wet pad at one extremity of the flow path.

Active propulsion of the sample through the device may be achieved by use of a pumping mechanism which may be external or internal (integrated), e.g., an external pump and/or a MEMS-style pump, via centrifugation such that the liquid is propelled in the desired direction, by applying a negative pressure at the end of the device (e.g., using a syringe, evaporation patch or vacuum or capillary suction-pad), by pressing the liquid forward through the device by a positive pressure applied by, e.g., a syringe or syringe-like device, or by pressing an enclosed compressible liquid compartment with the force of, e.g., the fingers, or by electro-osmotic or electro-kinetic flow.

The capture agent can be any molecule or matrix which can selectively bind one or several analytes. Preferably the capture agent has a high affinity and specificity for the molecular species to be detected and/or quantified, with little or no cross-reactivity to other species.

In a preferred embodiment, the capture agent is a protein, notably an antibody or a fragment thereof, a receptor, an enzyme, or a protease. In another embodiment, the capture agent is an oligonucleotide or polynucleotide, aptamer, an artificially generated protein-binding scaffold, or a phage. In another embodiment, the capture agent is a peptide, oligo- or polysaccharide, or phospholipid. In another embodiment, the capture agent is a small molecule, a drug, a non-biological polymer or a supramolecular structure. If the analyte is known to have an affinity to another species, that other species can potentially be used as the capture agent. The depletion flow path may also be coated with several different capture species which are specific for the same or different analytes. This expedient can be used to increase the binding strength to the analyte, to probe for different epitopes of an analyte, or to measure several different analyte species within the same flow path.

In the systems or devices of the invention, the capture agents are adhered or bound to a solid substrate. The substrate may consist of a material of construction of the device, as described above, and may include a coating or gel. Adherence or placing of the capture agent on the depletion flow path can be achieved through various methods as known in the art, for example by binding the capture species to the substrate using methods such as those described in U.S. Pat. Nos. 6,329,209, 6,365,418, 6,576,478, 6,406,821, 6,475,808, 6,630,358, and 6,682,942, which are hereby incorporated herein to the extent that their disclosures are not inconsistent with the disclosure herein.

The capture agent can be specifically or non-specifically immobilized on the surface of the depletion flow-path. It can be integrated into the material of the flow path itself, it can be formed at the surface of the flow path or it can be indirectly attached to the surface of the flow path by one or several interface layers. Examples of such interface layers include organosilanes, alkanethiol-based or disulfide-based self-assembled monolayers, copolymers, inorganic layers, bifunctional crosslinkers, hydrogels or passively adsorbed proteins such as avidin or albumin species.

The flow-path surface can further be modified with a plurality of different molecular species, e.g., by using certain moieties to promote the binding of the analytes and others to prevent the non-specific adsorption of other components that may be present in the sample. This approach can also be used to dilute the density of capture agents on the surface, e.g., to adjust the dynamic range in which the assay is operating.

The capture agent density, or the relative abundance of capture agent, can be deposited along the length of the flow path in a linear or nonlinear gradient. The capture agent density could be in an exponential, increasing gradient along the depletion path length. This method can be used to extend the dynamic sensitivity range of the test device. The capture agent can also be deposited in sequential or parallel patches of varying density.

Alternatively, the capture agents can be deposited in the flow-path in discrete areas, using e.g. a micro-arraying tool, ink-jet printer, spray, pin-based contact printing or screen-printing method. The regions between discrete capture agent areas can be modified with non-binding molecular species or blocked with methods known in the art (e.g. using BSA solutions in the case of protein depletion assays, etc.).

The capture agents can be further deposited in nano-, micro- and macro-patterns, allowing for e.g. diffractometric readout or by other optical interference mechanisms. The capture agents can further be deposited in such patterns as to prevent clogging or crowding of the flow path by immobilized analyte.

It may be necessary to keep the device, or at least that portion of it containing the capture agent, dry, moist, lyophilized or otherwise preserved in order to maintain its activity during storage. Possible means for such preservation include lyophilization of the capture agents or the use of preservative solutions (e.g., protein- or sugar-based solutions) first applied, and then dried, onto the capture layer. Alternatively the device can also be kept or stored fully pre-loaded with a storage, preservation or pre-wetting fluid.

Analyte capturing may also be done by mixing or exposing the analyte capture agent to the analyte before the sample is run through the flow path. In this method the capture agent has a secondary tag or epitope which can then be captured by a second capture agent in the flow path while the analyte is bound to its capture agent One possible embodiment of this approach is the use of analyte-specific antibodies linked to biotin, with the depletion flow path coated with avidin species to capture the biotinylated antibodies. The non analyte-bound capture agent can be removed from the sample, e.g., through a size-excluding material in a pre-section to the depletion flow path or by selectively binding that capture fraction to a species behind a size-selective membrane (e.g., a dialysis membrane of selected pore size). It is also possible to use a cascade of capture agents (e.g., the device can contain a sandwich immunoassay with multiple interaction partners).

In order to visualize the depletion length or the sections of the flow path which contain bound analyte molecules (or do not contain, for example, if the assay is a competitive assay), different labeling or detection methods can be used. In one embodiment, the device employs a label-free method in which the presence or absence of captured molecules is visible without a label. Such detection can be accomplished if the analytes are large, e.g., cells or other particles, or if the analyte is stained or intrinsically colored such that it can be detected without additional label or stain. A magnifying device such as a lens or microscope may be needed to carry out the readout.

In one preferred embodiment labeled detection antibodies are used. They are allowed to bind to the analyte either before or after the sample is flushed over the depletion flow path. The labeled antibodies specifically bind to the analyte and make it detectable by the unaided eye, calorimetrically or by other optical methods such as fluorescent or colorimetric readers, depending on the type of label used. The labels on the detection species can be any moiety typically used in the art for such purposes, including fluorescent dyes, colored beads or microspheres, gold or silver or other nanoparticles, radioactive species, quantum-dots, radio-tags, Raman tags, chemiluminescent labels, organic stains, etc.

Alternatively, any enzyme-amplified detection mode can be used, as is typically implemented for the readout of microtiter plate-based assays. Possible embodiments of such detection species are antibodies linked to, e.g., peroxidases, phosphatases or dehydrogenases, which are used in combination with an appropriate calorimetric enzyme substrate. For instance, an HRP-linked detection antibody can be used in combination with TMB as the enzyme substrate, leading to a blue substrate product in those depletion flow path areas which contain the captured analyte.

In a preferred embodiment, the areas containing analytes with bound detection species become visible to the unaided eye and can easily be distinguished from the areas with significantly less, or no, bound analyte. For cells, for instance, non-specific or specific cytoplasmic labeling, non-specific or specific cell membrane labeling with fluorophores of colored beads, or non-specific or specific nuclear labeling with fluorophores of colored beads, can be used. Cell labeling can be done in a separate reaction compartment or channel, or together with other processes in a reaction compartment or channel.

The devices of the invention may include elements that enhance the ability to read out the depletion length (e.g. readout contrast). Such elements include materials of different optical clarity and reflectivity, polarizing elements, micro-lens arrays, micro-lenses, LED lights, etc. Several different detection species may be run in parallel through a flow path or through parallel flow paths to detect various analytes in parallel. The detection species may have to exhibit different colors or optical properties so as to allow the unaided eye or the detection unit to differentiate between the different detection species.

The method can be used to determine the presence or absence of a specific analyte (non-quantitatively) in a sample, or to quantify it relative to an internal, external or factory-calibrated standard.

The binding of the analyte to the capture agent may be covalent, ionic, electrostatic or through any other type of interaction. The binding may be reversible or irreversible and may necessitate that the readout is done within a predefined time interval after starting or ending the depletion assay run.

Certain embodiments of the invention may use electronic and/or optical read-out devices to perform the quantification of the assay readout. Such devices can include hand-held devices connected to microprocessors, specifically designed analytical instrumentation and readout devices which can transmit the readout information wirelessly to data receiving/distribution centers.

The depletion length or area readout can be done by any method known in the art. These include reading the depletion length or area using electrochemical methods, by measuring the change in electrical conductivity (along the depletion flow path or orthogonal thereto), or by detecting a change in optical parameters (e.g., using a photosensor array positioned in close proximity to the flow path). Other test-result readout modes may include diffractometric methods in which the capture molecules are arranged in defined patterns on the flow-path surface, forming a diffraction grating which can be read by a laser, and methods based on using liquid crystal technology to visualize the depletion length (e.g. linking the detection species to optically active molecules which change the polarization of light and can thus be read via liquid crystal display technology). However, especially for use in resource-poor areas, a preferred embodiment of the invention allows the readout by the unaided eye, without the need for any electronic or external detection instrumentation.

The readout may be done relative to a lateral reference ruler or a colored or gray-scale structure reference printed or included on or in the depletion flow path. Alternatively a reference scale may be separately provided with the test device.

The devices of the invention may include positive or negative control areas or zones which may be included in parallel to, before, after or within the depletion flow path. Such control zones may, e.g., be used to verify that the sample liquid completely flows through the depletion flow-path, or that certain assay reagents are still active when the assay is carried out, or that the calibration of the device is still accurate. Embodiments of such control areas may include areas coated with reagents that change in color when wetted, or areas containing immobilized antibodies specific to molecules in the sample, or to the detection species, or to reagents contained in the assay kit. The device may further incorporate a reference sample which can be run in a separate depletion flow path of the device.

Access to the test results can be accomplished by several means. In one embodiment the flow path is exposed to the atmosphere and can be read directly. In another embodiment a transparent cover is placed over the flow path for protection against contamination. Again, the readout can be taken directly by the unaided eye or by an instrument. In another embodiment the flow path is covered, but a transparent “window” is provided over that area of the flow path that would show a labeled depletion end region at a certain concentration. Such a device could be used for readily available “yes/no” determination of whether a given analyte is present in a sample at a certain concentration, for instance the legal maximum or minimum concentration for a particular drug. If the analyte is present in the sample at that concentration the label will be detectable through the window; otherwise it would not be detected.

The assay time typically is in the range of from about 30 seconds to about 30 minutes. In some embodiments the assay may take only a few seconds to a few minutes to run. In other embodiments, however, the assay may take several hours or even days. The assay may run on its own once the sample has been introduced, or one or more user intervention steps may be required during the assay. The assay may also include features which direct the user to perform certain tasks after receiving specific signals from the device. Such tasks may e.g. include pressing certain assay cartridge features, e.g., to inject an enzyme substrate into the depletion flow path after running an assay detected by an enzyme amplified detection mode.

Good shelf life stability can be achieved by implementing a liquid reagent-less test strip design. In such a device chemicals or biochemicals may be immobilized on surfaces and then preserved by preserving agents such as trehalose. After preservation, the strips are dried and then sealed into a pouch with or without a drying agent (desiccant pouch) and/or an inert gas filling.

A typical sequence of events in running the depletion assays of the invention would be as follows:
1) Insertion of the sample
2) Optionally, sampling of a defined sample volume by an upstream cartridge feature
3) Optionally sample pre-treatment, e.g. to remove unwanted species from the sample
4) Optionally labeling of the analyte in solution by soluble labeled antibodies
5) Flowing the sample through the depletion flow path.
6) Optional labeling of the analyte in solution by soluble labeled antibodies
7 Reading the depletion length of the analyte in the depletion flow path.
8 Comparing the depletion length to an integrated calibration standard to determine the initial concentration of the analyte in the sample.
9) Discarding the device.

FIGS. 5 and 6 show a system according to one embodiment of the invention that is suitable for determining CD4 cell count in a subject's blood sample, for use in identifying and monitoring the immune status of HIV-positive patients. In this approach, whole blood samples are funneled through a channel architecture integrated into a test strip having walls coated with one or more specific anti-CD4 capture agents. As the blood flows through the channel, CD4 cells adhere to the channel walls, thereby depleting the blood sample from CD4 cells not crossing a pre-calibrated boundary with an analyte depletion end region being detectable at a pre-calibrated location if the cell count is below a certain level.

The device shown in FIGS. 5 and 6 is made of plastic. It integrates all the necessary sample pre-treatment reaction steps and will allow visual determination of the T-cell count directly from the test strips. Because of the extreme shelf-life conditions that would be encountered in tropical or arid areas, liquid-based protein solutions should be avoided in devices for use under such conditions. Thus, such devices utilize dried, but preserved (protein) reagents on the strips. Such dry reagents can be engineered to have excellent storage stability and assay performance. The strips will also integrate a positive control for verifying the correct functioning of the T-cell test. A waste reservoir which will allow hermetically sealing of the device will allow the disposal of the devices after use without the risk of infecting personnel from blood samples.

FIG. 5 shows a schematic overview of the key elements of the device, indicated generally as 51. A defined amount of blood drawn from a finger-prick is either injected into the strip through a port 53, or, alternatively, a finger-pricking element can be integrated directly into the plastic device. The blood sample is then pushed through the different reagent chambers e.g. by centrifugation (e.g., a small hand-driven centrifuge) or via other mechanical mechanisms known in the art. After sampling, a defined blood volume (constant volume mechanism), is transported into a first reaction chamber 55 to remove any potentially interfering non-T-cell species from the sample solution (e.g. by anti-CD14 capture antibodies immobilized onto the walls). The blood sample is then transferred into an optional second reaction chamber 57, in which the T-cells can be labeled for easier visual detection further downstream (e.g., by cytoplasmic staining). The labeled T-cells then reach a long depletion channel 59 coated with anti-CD4 capture agents. By careful optimization of the binding capacity, microfluidic properties and surface area in that channel, the CD4⁺ T-cells will quantitatively deplete from solution by binding to the flow path surface. A reference guide 61 is provided to ascertain concentration of the cells in the sample. The length of the depletion channel that is visibly coated with labeled CD4⁻ T-cells will be in a pre-calibrated relation to the T-cell count. Further downstream, a small window 63 with, e.g., antibodies against the cell dye, will allow verification that the test-strip is still functional (positive control). Ultimately, the used blood sample reaches a waste reservoir 65 at the end of the test strip.

A semi-quantitative readout based on defined cut-offs for the CD4⁺ T-cell count can be achieved by directly integrating a visual readout 61 into the test strip, without the need for a separate reader. The cell quantification approach in this method is based on sequentially depleting all the CD4⁺ cells present in a defined blood volume onto the walls or surfaces of a microchannel or of material contained in it, and then determining the length of the channel that is coated with cells as a direct measure of the cell count. Compared to methods based on quantifying the intensity of e.g. labels previously attached to T-cells, this method is independent from the labeling efficiency, requires no separation steps and can be done using surface-attached capture molecules (no liquid reagents nor separation/lysis of the erythrocytes are needed).

FIG. 6 depicts how the cell count would be determined in devices of the invention. Through careful adjustment, the design in terms of internal dimensions and the binding capacity of the device relative to the diffusion rate of the cells and the flow rate of the sample will allow the formation of a very sharp boundary between areas with and without cells attached to the walls of the microchannel. The depletion border or end region 62 is clear and, using the guide 61, indicates the concentration of CD4⁺ cells in the sample. The positive control 63 shows that the device functioned properly, and waste sample has been collected in reservoir 65.

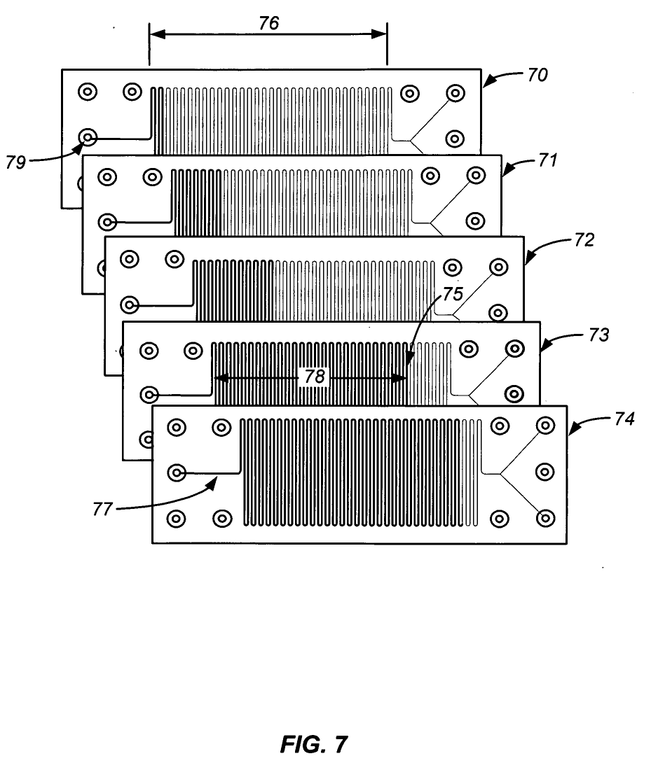

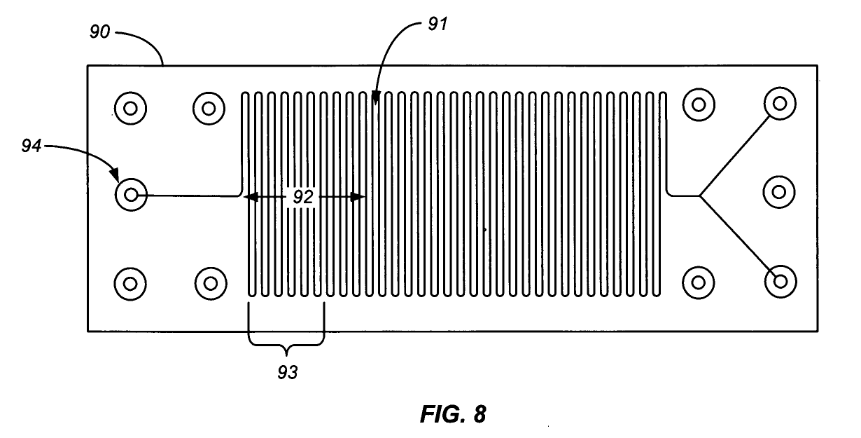

FIGS. 7 and 8 depict work done in calibrating an assay strip and device such as that shown in FIGS. 5 and 6.

FIG. 7 is a photograph of five depletion assay chips specific for Human-IL-10 cytokine analyte, after having been run with five different concentrations of Human-IL-10 analyte, showing an increasing depletion edge length according to the IL-10 analyte concentrations run in those respective chips. This demonstrates the protein depletion assay principle using an immunoglobulin sandwich assay in one embodiment having glass microchannel chips (70, 71, 72, 73, 74) each containing a 60-cm long, curved depletion channel (77) with a channel inner dimension of about 150 micrometers and channel inlets (79) and a defined, effective flow-path total length (76).

The glass channels were oxygen-plasma activated, then homogeneously coated with a biotinylated PLL-PEG-biotin-30% copolymer (40 μl at 1 mg/ml in 10 mM Hepes Buffer pH 7.4 for 30 min). After washing with 60 μl PBS pH 7.4, the channels were incubated with streptavidin (1.66 μM; 40 μl for 10 min) and washed again (PBS pH 7.4, 60 μl). Afterwards the channels were incubated with capture agent (anti-human-IL10 antibody, 40 μl at 1 μM; overnight) and then blocked/washed with 15% fetal bovine serum (FBS) in PBS pH 7.4, 60 μl. After that, the chips were run in depletion mode by flowing 6 μl of different concentrations of human-IL-10 analyte sample through the channels at a flow rate of 0.3 μl/min (syringe pump). During that process, the analyte binds to the anti-IL-10 antibodies on the channel walls in depletion mode. After washing with PBS pH 7.4, incubation of detection antibody (anti-human-IL-10 antibody labeled with phycoerythrin, 40 μl at 100 nM in 15% FBS for 30 min), and again washing with PBS pH 7.4 (40 μl), pictures were taken of the chips in a fluorescent gel-reader apparatus equipped with a 360 nm wavelength UV black-light table and an ethidium bromide-specific filter in front of a camera. A clear depletion edge (e.g. 75) is visible on the different chips defining a specific analyte depletion length (e.g. 78), which correlates with the analyte amount (concentration) in the samples. The analyte concentrations run on the different chips were: Chip 70: 200 nM; Chip 71: 400 nM; Chip 72: 600 nM; Chip 73: 800 nM; Chip 74: 1000 nM. The designation 76 shows the total effective depletion channel length within which the sample depletion edge/length is detected.

FIG. 8 is a photograph of a depletion assay chip (90) having a sample inlet 94, demonstrating reader-less readout of the depletion length. This chip was run with biotin (PLL-PEG-biotin 30% copolymer) immobilized on the flow path walls as capture agent; streptavidin conjugated to alkaline phosphates enzyme (SA-AP) was used as the analyte. After running a specific volume and concentration of SA-AP in depletion mode through the chip, a colorimetric substrate for the AP (BCIP) was injected into the fluidic channel. In those flow path sections containing immobilized analyte on the channel walls, the enzyme transforms the transparent enzyme-substrate BCIP into a dark-colored, insoluble product. The depletion edge (91) thus becomes visible as the transition from dark to transparent in the channel, which can be seen by the unaided eye. The corresponding depletion length is shown as 92. Very high enzyme concentrations on the channel walls can lead to over-saturation of the enzyme product, making it turn transparent again, which could explain why some of the depletion length becomes transparent again (93).

The foregoing descriptions are offered primarily for purposes of illustration. Further modifications, variations and substitutions that still fall within the spirit and scope of the invention will be readily apparent to those skilled in the art. All such modifications coming within the scope of the appended claims are intended to be included therein.

All publications, patents, and patent applications cited herein are hereby incorporated by reference in their entirety for all purposes, except to the extent inconsistent with the disclosure herein.

What is claimed is:

1. A system for determining one or more analytes in a sample that comprises a fluid, the system comprising a solid substrate having a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path or paths, the flow path or paths being designed so that the one or more analytes are depleted from the sample and bound in a non-linear manner to the portion of the flow path or paths containing immobilized capture agent or agents, producing an analyte depletion end region for each analyte between the beginning and the terminus of the analyte determination flow path.

2. A system according to claim 1 for determining a biological or chemical material in the sample

3. A system according to claim 1 for determining a cellular material in the sample.

4. A system according to claim 1 for determining a chemical material in the sample.

5. A system according to claim 1 in which the flow path comprises one or more sections.

6. A system according to claim 5 in which the flow path comprises one or more chambers separated by passageways through which the sample can flow.

7. A system according to claim 6 in which the passageways separating the chambers are substantially free of the one or more capture agents.

8. A system according to claim 1 further comprising means for propelling the sample along the flow path.

9. A system according to claim 1 in which the flow path comprises a porous material.

10. A system according to claim 1 in which at least the major portion of the length of the flow path contains the capture agent.

11. A system according to claim 1 wherein the one or more analytes are labeled.

12. A system according to claim 11 wherein the analyte is a cellular, chemical or biochemical material and the label is selected from the group consisting of beads, colloids, liposomes, microspheres, dyes, radioactive labels, IR-labels, electrochemical labels, fluorescent labels, Raman labels, luminescent labels, enzymatic labels, quantum-dots and organic stains.

13. A system according to claim 11 in which the analyte is a cellular material and the label comprises a stain.

14. A system according to claim 1 further comprising means for selectively viewing the detectable analyte depletion end region.

15. A system according to claim 1 for determining the concentration of the analyte in the sample.

16. A system according to claim 15 in which the location of the analyte depletion end region in the flow path is indicative of the concentration of the analyte in the sample.

17. A system according to claim 1 further comprising structures in the flow path that increase the surface area available to provide capture agents and/or facilitate mixing of substances in the flow path.

18. A system according to claim 1 wherein the capture agents are selected from the group consisting of antibodies, antibody fragments, aptamers, phages, protein-binding scaffolds, natural or artificial binding partners, and combinations of two or more of the foregoing.

19. A system according to claim 1 further comprising positive and/or negative control areas.

20. A system according to claim 1 further comprising a calibrated measure to relate the analyte depletion end region for each analyte to the amount or concentration of said analyte in the sample.

21. A system according to claim 1 further comprising a detector disposed along or adjacent to the flow path for detecting the depletion length of the analyte.

22. A method for determining one or more analytes in a sample that comprises a fluid, the method comprising introducing the sample into a system or device comprising a solid substrate having a sample inlet or inlets and one or more analyte determination flow paths, each analyte determination flow path comprising a defined beginning and a defined terminus and comprising at least one capture zone containing a capture agent for an analyte, the capture agent or agents being immobilized along a portion of the flow path, the flow path or paths being designed so that the one or more analytes are depleted from the sample and bound in a non-linear manner to the portion of the flow path or paths containing immobilized capture agent or agents, producing an analyte depletion end region for each analyte between the beginning and the terminus of an analyte determination flow path, causing the sample to flow through the flow path and, based on the location of each analyte depletion end region, determining the presence and/or concentration of said analyte in the sample.

23. A method according to claim 22 comprising determining the concentration of analyte in the sample.

24. A method according to claim 22 for determining a cellular material in the sample.

25. A method according to claim 22 for determining a chemical material in the sample.

26. A method according to claim 22 in which the system further comprises means for selectively viewing the analyte depletion end region.
